(19) United States
(12) Patent Application Publication
(10) Pub. No.: US 2026/0041315 A1
(43) Pub. Date: Feb. 12, 2026

(54) SYSTEM

(71) Applicant: FUJIFILM Corporation, Tokyo (JP)

(72) Inventor: Hiroaki TABE, Ashigarakami-gun (JP)

(73) Assignee: FUJIFILM Corporation, Tokyo (JP)

(21) Appl. No.: 19/283,520

(22) Filed: Jul. 29, 2025

(30) Foreign Application Priority Data
Aug. 9, 2024 (JP) 2024-134019

(51) Int. Cl. A61B 1/06; A61B 1/00; G16H 40/63

(52) U.S. Cl. CPC A61B 1/0661; A61B 1/00009; A61B 1/00043; A61B 1/0655; G16H 40/63

(57) ABSTRACT

A system includes: a light source device including a light emitting unit and a sensor that measures a characteristic value of the light emitting unit, and configured to supply light emitted from the light emitting unit to an endoscope; and a processor. The processor is configured to: in a first case where the endoscope is not connected to the light source device, or in a second case where the endoscope is connected to the light source device and is in a non-use state, perform light emission control of causing the light emitting unit to emit light under a first condition; acquire, from the sensor, the characteristic value of the light emitting unit that has emitted light under the light emission control; and determine a state of the light emitting unit based on the characteristic value and a reference value.

### CROSS-REFERENCE TO RELATED APPLICATION

This application is based on and claims priority under 35 USC 119 from Japanese Patent Application No. 2024-134019 filed on Aug. 9, 2024, the contents of which are incorporated herein by reference.

### BACKGROUND OF THE INVENTION

### 1. Field of the Invention

The technology of the present disclosure relates to a system.

### 2. Description of the Related Art

WO2016/56477A discloses a light source device including a plurality of light emitting diodes (LEDs) that emit illumination light of different colors for illuminating a subject, an LED driving unit that generates a drive current for each LED, a light sensor that detects an illuminance value of the illumination light of each LED, a memory that stores, as a table, illuminance values in a predetermined range corresponding to the drive current when each LED normally emits light, and a controller that, by referring to the table, determines whether or not an illuminance value of any LED detected by any light sensor is within the predetermined range, and, in a case where it is determined that the illuminance value of any LED is not within the predetermined range, detects an abnormality in any light sensor or an abnormality in any LED.

### SUMMARY OF THE INVENTION

In the technology of the present disclosure, a system that can determine a state of a light emitting unit in a light source device connected to an endoscope is provided.

A system according to an aspect of the present disclosed technology comprises: a light source device including a light emitting unit and a sensor that measures a characteristic value of the light emitting unit, and configured to supply light emitted from the light emitting unit to an endoscope; and a processor, the processor is configured to: in a first case where the endoscope is not connected to the light source device, or in a second case where the endoscope is connected to the light source device and is in a non-use state, perform light emission control of causing the light emitting unit to emit light under a first condition; acquire, from the sensor, the characteristic value of the light emitting unit that has emitted light under the light emission control; and determine a state of the light emitting unit based on the characteristic value and a reference value.

In the technology of the present disclosure, it is possible to determine a state of a light emitting unit in a light source device connected to an endoscope.

### BRIEF DESCRIPTION OF THE DRAWINGS

FIG. 1 is an external view showing an endoscope apparatus 2 according to an aspect of the technology of the present disclosure.

FIG. 2 is a schematic view showing an example of an internal configuration of a light source unit 30 shown in FIG. 1.

FIG. 3 is a schematic view showing an example of an internal configuration of a light emitting unit 31 shown in FIG. 2.

FIG. 4 is a flowchart for describing a processing example (1) of a controller 40.

FIG. 5 is a flowchart for describing a specific example of step S13 and step S14 shown in FIG. 4.

FIG. 6 is a flowchart for describing a processing example (2) of the controller 40.

FIG. 7 is a flowchart for describing a processing example (3) of the controller 40.

### DESCRIPTION OF THE PREFERRED EMBODIMENTS

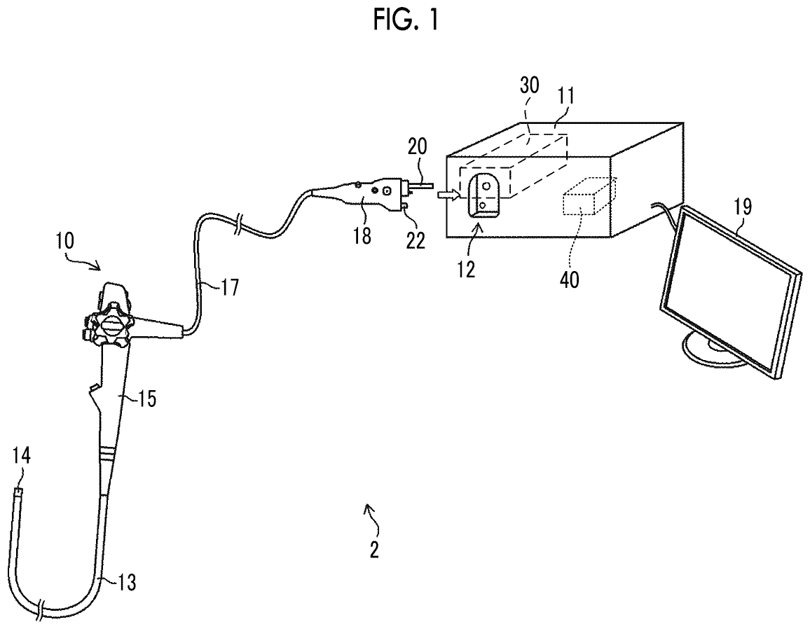

FIG. 1 is an external view showing an endoscope apparatus 2 according to an aspect of the technology of the present disclosure. As shown in FIG. 1, the endoscope apparatus 2 comprises an endoscope 10, a control device 11, and a display device 19. The control device 11 includes a light source unit 30 and a controller 40, and the controller 40 comprehensively controls the entire endoscope apparatus 2 in accordance with an operation of an operator input from an input device (an operation switch, a keyboard, a mouse, or the like). The controller 40 includes a processor and a memory. The controller 40 may be provided in a device different from the control device 11. The control device 11 constitutes a system comprising a light source device and a processor.

The endoscope 10 is an example of a flexible endoscope, and includes a flexible insertion part 13 to be inserted into a body cavity of a patient, an operation part 15 provided at a proximal end portion of the insertion part 13, a universal cord 17 provided at the operation part 15, and an endoscope connector 18 provided at an end part of the universal cord 17 and connected to the connector 12 of the control device 11. The endoscope 10 is not limited to a flexible endoscope, and may be another type of endoscope, such as a rigid endoscope.

An observation window, an illumination window, and the like are provided on a distal end surface of the insertion part 13. A distal end part 14 that constitutes a distal end of the insertion part 13 is provided with an imaging unit including an objective optical system that forms an optical image of subject light from a part to be observed taken in through the observation window, and an imaging element that converts the optical image formed by the objective optical system into an image signal. The imaging element is, for example, a charge coupled device (CCD) image sensor, a complementary metal oxide semiconductor (CMOS) image sensor, or the like. The imaging unit is controlled by the controller 40.

The image signal output from the imaging unit is transmitted to the endoscope connector 18 by a transmission cable inserted into and disposed in the endoscope connector 18 through the inside of the insertion part 13, the operation part 15, and the universal cord 17. The controller 40 performs display control of processing the image signal to generate an image for display and outputting the image to the display device 19 to display the image. For example, the controller 40 starts the display control in a case where an examination start button provided in the input device is operated, and ends the display control in a case where an examination end button provided in the input device is operated.

A light emitting unit of a light guide that transmits light to be emitted to the part to be observed from the illumination window is disposed in the distal end part 14. The light guide is inserted into and disposed in the endoscope connector 18 through the inside of the insertion part 13, the operation part 15, and the universal cord 17. The light guide rod 20 connected to the light guide is provided to protrude from the endoscope connector 18.

The operation part 15 includes an angle knob for adjusting the orientation of the distal end surface of the insertion part 13 in the vertical and horizontal directions, an air/water supply button for ejecting air and water from the distal end surface of the insertion part 13, a release button for recording a still image of the captured image, and the like. The orientation of the distal end surface of the insertion part 13 is adjusted by bending a bendable part provided in the vicinity of the proximal end side of the distal end part 14.

The universal cord 17 is covered with an outer wall part that is tubular and elongated, and has flexibility. The transmission cable and the light guide described above, which are inserted into and disposed in cavity parts inside the insertion part 13 and inside the operation part 15, the air/water supply tube, and the like are inserted into and disposed in a pipe inside the outer wall part.

The endoscope connector 18 is connected to the connector 12 of the control device 11. The endoscope connector 18 and the connector 12 perform the supply of power from the control device 11 to the endoscope 10, the transmission of the image signal from the endoscope 10 to the control device 11, and the transmission and reception of the control signal between the endoscope 10 and the control device 11, preferably in a noncontact manner without the physical connection of the electrical wires. The supply of power from the control device 11 to the endoscope 10, the transmission of the image signal from the endoscope 10 to the control device 11, and the transmission and reception of the control signal between the endoscope 10 and the control device 11 may be performed by a physical electrical wire.

The control device 11 comprises a light source unit 30 which is one aspect of a light source device. The light source unit 30 has, for example, a plurality of light emitting units including a semiconductor device such as a laser diode or a light emitting diode. In a case where the endoscope connector 18 is mounted on the connector 12 of the control device 11, the light guide rod 20 of the endoscope 10 is connected to the light source unit 30 via the connector 12, and the light emitting unit of the light source unit 30 and the light guide rod 20 are aligned with each other. As a result, the light from the light source unit 30 is transmitted to the distal end part 14 of the endoscope 10 via the light guide rod 20 and the light guide.

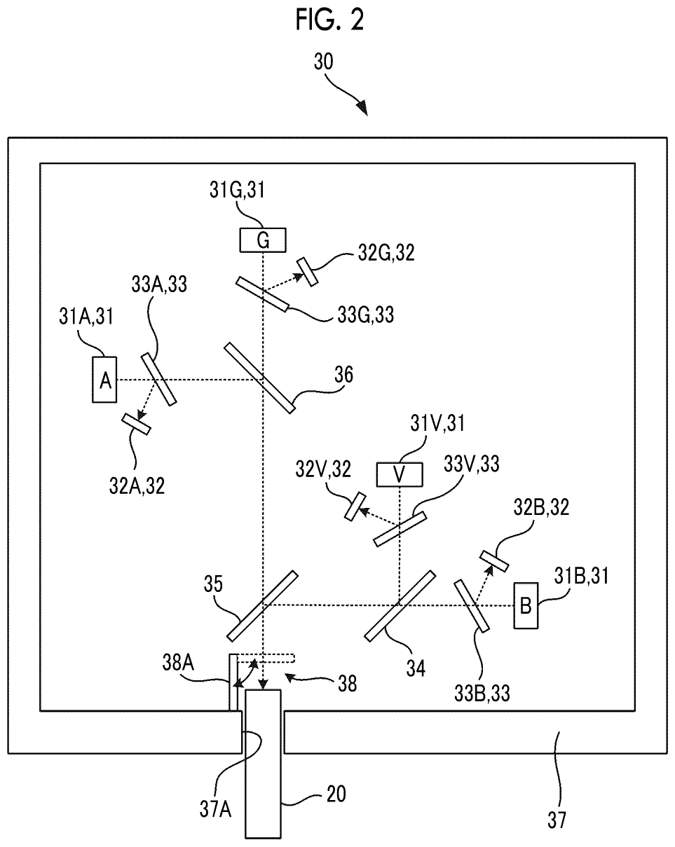

FIG. 2 is a schematic view showing an example of an internal configuration of the light source unit 30 shown in FIG. 1.

The light source unit 30 includes, inside a housing 37, a plurality of (four in example shown in FIG. 2) light emitting units 31 that generate light of different colors, an optical member (a dichroic mirror 34, a dichroic mirror 35, and a dichroic mirror 36) configured to introduce one or more types of light generated by the four light emitting units 31 into a light guide of the endoscope 10, and four sensors 32 configured to measure characteristic values of each of the four light emitting units 31. In the example of FIG. 2, one sensor 32 is provided corresponding to each of the four light emitting units 31.

The housing 37 is provided with an opening 37A through which the light guide rod 20 of the endoscope 10 can be inserted. The housing 37 is provided with a shielding mechanism 38 including a movable shutter member 38A and a drive unit that drives the shutter member 38A.

The four light emitting units 31 and the shielding mechanism 38 are controlled by the controller 40. The sensor 32 can measure the light emission amount of the light emitting unit 31 as the characteristic value of the corresponding light emitting unit 31, and is composed of a light-receiving element such as a photodiode or a photoresistor.

The four light emitting units 31 generate light in four wavelength ranges having different central wavelengths. The four light emitting units 31 include, for example, a light emitting unit 31B that generates light in a blue wavelength range (hereinafter, referred to as B light), a light emitting unit 31V that generates light in a violet wavelength range (hereinafter, referred to as V light), a light emitting unit 31G that generates light in a green wavelength range (hereinafter, referred to as G light), and a light emitting unit 31A that generates light in an amber (or red) wavelength range (hereinafter, referred to as A light). The type and the number of colors of the light generated by the light source unit 30 are not limited to the present embodiment.

The light emitting unit 31G, the dichroic mirror 36, the dichroic mirror 35, and the opening 37A are arranged in a straight line in this order. The G light emitted from the light emitting unit 31G transmits through the dichroic mirror 36 and the dichroic mirror 35 and is incident on the light guide rod 20 inserted into the opening 37A.

In the dichroic mirror 36, a light reflecting surface faces the left side in the drawing and is provided to be inclined at an angle of 45 degrees with respect to a path of G light. The light emitting unit 31A is provided at a position facing a light reflecting surface of the dichroic mirror 36. The A light emitted from the light emitting unit 31A is reflected by the dichroic mirror 36, is incident on the dichroic mirror 35, and is transmitted through the dichroic mirror 35 to be incident on the light guide rod 20.

In the dichroic mirror 35, a light reflecting surface faces the right side in the drawing and is provided to be inclined at an angle of 45 degrees with respect to a path of G light. The light emitting unit 31B is provided at a position facing a light reflecting surface of the dichroic mirror 35. The dichroic mirror 34 is provided between the light emitting unit 31B and the dichroic mirror 35. The B light emitted from the light emitting unit 31B passes through the dichroic mirror 34, is incident on the dichroic mirror 35, is reflected from the dichroic mirror 35, and is incident on the light guide rod 20.

In the dichroic mirror 34, a light reflecting surface faces the upper side in the drawing and is provided to be inclined at an angle of 45 degrees with respect to a path of B light. The light emitting unit 31V is provided at a position facing a light reflecting surface of the dichroic mirror 34. The V light emitted from the light emitting unit 31V is reflected from the dichroic mirror 34, is incident on the dichroic mirror 35, is reflected from the dichroic mirror 35, and is incident on the light guide rod 20. The light source unit 30 is configured to supply, to the light guide of the endoscope 10, combined light in which two or more of the G light, A light, B light, and V light are combined.

The combined light travels from the dichroic mirror 35 to the opening 37A or the light guide rod 20 inserted into the opening 37A. The shielding mechanism 38 can be switched between a shielding state and a non-shielding state. The shielding state is a state in which the shutter member 38A is inserted between the dichroic mirror 35 and the opening 37A, and at least a part of the combined light is prevented from being incident into the opening 37A or the light guide rod 20 inserted into the opening 37A. The non-shielding state is a state in which the shutter member 38A is retracted from between the dichroic mirror 35 and the opening 37A, and all the combined light can be incident on the opening 37A or the light guide rod 20 inserted into the opening 37A. The endoscope 10 may be attachable to the connector 12 only in a case where the shielding mechanism 38 is in the non-shielding state, or may be attachable to the connector 12 in any of the shielded state or the non-shielding state of the shielding mechanism 38.

The sensor 32 includes a sensor 32G that is provided corresponding to the light emitting unit 31G and is capable of measuring a light emission amount of the light emitting unit 31G, a sensor 32B that is provided corresponding to the light emitting unit 31B and is capable of measuring a light emission amount of the light emitting unit 31B, a sensor 32V that is provided corresponding to the light emitting unit 31V and is capable of measuring a light emission amount of the light emitting unit 31V, and a sensor 32A that is provided corresponding to the light emitting unit 31A and is capable of measuring a light emission amount of the light emitting unit 31A.

A half mirror 33G is provided between the light emitting unit 31G and the dichroic mirror 36. A part of the G light emitted from the light emitting unit 31G is reflected by the half mirror 33G to be incident on the sensor 32G, and the remaining part of the G light is transmitted through the half mirror 33G to be incident on the dichroic mirror 36.

A half mirror 33A is provided between the light emitting unit 31A and the dichroic mirror 36. A part of the A light emitted from the light emitting unit 31A is reflected by the half mirror 33A to be incident on the sensor 32A, and the remaining part of the A light is transmitted through the half mirror 33A to be incident on the dichroic mirror 36.

A half mirror 33V is provided between the light emitting unit 31V and the dichroic mirror 34. A part of the V light emitted from the light emitting unit 31V is reflected by the half mirror 33V to be incident on the sensor 32V, and the remaining part of the V light is transmitted through the half mirror 33V to be incident on the dichroic mirror 34.

A half mirror 33B is provided between the light emitting unit 31B and the dichroic mirror 34. A part of the B light emitted from the light emitting unit 31B is reflected by the half mirror 33B to be incident on the sensor 32B, and the remaining part of the B light is transmitted through the half mirror 33B to be incident on the dichroic mirror 34.

The sensor 32 need only be configured to receive a part of the light emitted from the corresponding light emitting unit 31, and is not limited to the configuration shown in FIG. 2. For example, a configuration may be adopted in which the dichroic mirror 34 reflects a part of the B light in a downward direction in the drawing, and the sensor 32B is provided on a reflection path of the part of the B light. In this case, the half mirror 33B is unnecessary.

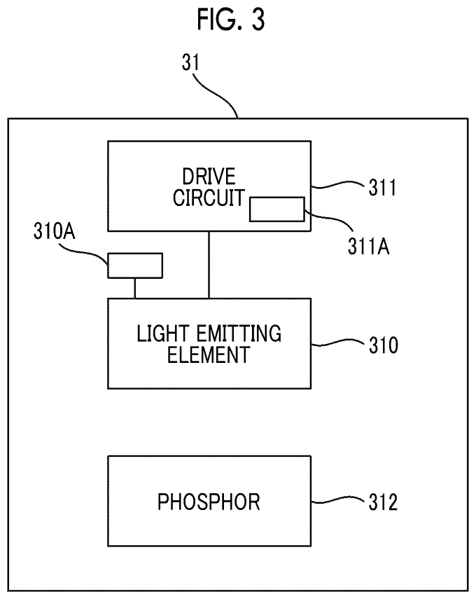

FIG. 3 is a schematic view showing an example of an internal configuration of a light emitting unit 31 shown in FIG. 2. The light emitting unit 31 comprises a light emitting element 310 composed of a semiconductor device such as an LED or a laser diode (LD), a drive circuit 311 that drives the light emitting element 310, and a phosphor 312 that emits fluorescence using light emitted from the light emitting element 310 as excitation light.

The phosphor 312 is provided depending on the color or the like emitted by the light emitting unit 31, and is not essential. In addition, although not essential, the drive circuit 311 may be provided with a measurement circuit 311A that measures a drive current or a drive voltage of the light emitting element 310. Similarly, although not essential, the light emitting unit 31 may be provided with a temperature sensor 310A such as a thermistor that measures the temperature of the light emitting element 310. The drive voltage of the light emitting element 310, the drive current of the light emitting element 310, and the temperature of the light emitting element 310 are each one of the characteristic values of the light emitting unit 31 including the light emitting element 310. The measurement circuit 311A and the temperature sensor 310A each configure a sensor corresponding to the light emitting unit 31.

The controller 40 may perform auto power control (APC), in which a measured value of the light emission amount of the light emitting unit 31 is acquired from a sensor 32 corresponding to the light emitting unit 31, and a drive current and a drive voltage of a light emitting element 310 of the light emitting unit 31 are controlled such that the measured value approaches a set light amount set in advance. As a result, even in a case where the light emission amount of the light emitting unit 31 fluctuates due to temperature drift or the like, the light emission amount of the light emitting unit 31 can be brought close to the target light amount.

In the first case or the second case, the controller 40 performs the light emission control of causing the light emitting unit 31 to emit light under the first condition, acquires the light emission amount of the light emitting unit 31 by the light emission control from the sensor 32, and performs processing (state determination processing) of determining the state of the light emitting unit 31 based on the acquired amount of emitted light (hereinafter, referred to as a measured light amount P1) and a reference value P2 of the amount of light based on the first condition for each of the four light emitting units 31. The state of the light emitting unit 31 includes performance related to the amount of emitted light (the degree of decrease in the amount of emitted light, which indicates how much the amount of emitted light actually obtained is decreased with respect to the target amount of emitted light). The decrease in the amount of emitted light may occur due to the deterioration over time, failure, or the like of the constituent elements included in the light emitting unit 31.

### First Example of First Condition

The first example is an example in which the controller 40 does not perform the APC control. In this case, the first condition includes setting the set value of the light emission amount of the light emitting unit 31 to any value that can be set by the light emitting unit 31 and driving the light emitting element 310 with a drive current and a drive voltage corresponding to the value. It is preferable that the first condition further includes bringing the shielding mechanism 38 into a shielding state and shielding at least a part of the light emitted from the light emitting unit 31 by the shutter member 38A.

In the first example, a value at the time of factory shipment of a measured light amount measured by the sensor 32 corresponding to the light emitting unit 31, the light emitting unit 31 being caused to emit light under the first condition, is known, and the value constitutes the reference value P2 described above. The degree of decrease in the light emission amount of the light emitting unit 31 can be determined by comparing the reference value P2 with a measured light amount P1 obtained at the time of performing light emission control. For example, in a case where the measured light amount P1 is significantly lower than the reference value P2, it can be determined that the light emitting unit 31 is in a state where deterioration or an abnormality has occurred.

### Second Example of First Condition

The second example is an example in a case where the APC control is performed. In this case, the first condition includes setting the set value of the amount of light emitted by the light emitting unit 31 to a value of 80% or more of the upper limit value (preferably, the upper limit value) that can be set by the light emitting unit 31. It is preferable that the first condition further includes bringing the shielding mechanism 38 into the shielding state.

In the second example, a value at the time of factory shipment of a measured light amount measured by the sensor 32 corresponding to the light emitting unit 31, the light emitting unit 31 being caused to emit light under the first condition, is known, and the value constitutes the reference value P2 described above. In the second example, in a state where a predetermined time has elapsed from light emission control of the light emitting unit 31 under the first condition (a state in which the light emission amount has stabilized by the APC control), a measured light amount P1 measured by the sensor 32 corresponding to the light emitting unit 31 is compared with a reference value P2, thereby enabling accurate determination of a degree of decrease in the light emission amount of the light emitting unit 31. For example, in a case where the difference between the reference value P2 and the measured light amount P1 is large, which is originally supposed to be close to the reference value P2 and the measured light amount P1, it can be determined by the APC control that the light emitting unit 31 is in a state where deterioration or an abnormality has occurred.

In a case where the APC control is performed, in a case where the set value of the light emission amount of the light emitting unit 31 in the first condition is excessively low, the reference value P2 and the measured light amount P1 may approach each other by adjusting the drive current or the drive voltage of the light emitting element 310 by the APC control even in a case where the light emitting unit 31 is in a state where deterioration or an abnormality has occurred. By sufficiently increasing the set value in the first condition, a decrease in the light emission amount of the light emitting unit 31, which cannot be adjusted by the APC control, can be detected. In particular, in a case where the set value is the upper limit value, even if APC control is performed, a decrease in the light emission amount of the light emitting unit 31, as compared with the amount at the time of factory shipment, can be accurately determined.

In the first case, the endoscope 10 is not connected to the light source unit 30. The controller 40 detects whether or not the endoscope 10 is connected to the light source unit 30 by using, for example, a mechanical sensor provided in the connector 12, or a circuit detecting electrical connection between the endoscope 10 and the connector 12.

The second case is a case where the endoscope 10 is connected to the light source unit 30 and the endoscope 10 is in a non-use state. The non-use state of the endoscope 10 includes a state in which the display control is not executed by the controller 40 and a state in which power is not supplied from the control device 11 to the endoscope 10.

The state in which the display control is not executed includes a state before the display control is started (that is, before the examination starts) or a state after the display control is ended (that is, after the examination ends) in a state in which the endoscope 10 is connected to the connector 12 and power is supplied from the control device 11 to the endoscope 10.

Although the endoscope 10 is connected to the connector 12, in a state where power is not supplied from the control device 11 to the endoscope 10, the display control is naturally not executed. Therefore, this state can also be said to be one of the states in which the display control is not executed.

In some endoscope apparatuses 2, in a case where the endoscope 10 is connected to the connector 12 and power is supplied from the control device 11 to the endoscope 10, the display control may be started regardless of the operation of the examination start button. In this case, it is assumed that the endoscope 10 is locked to a hook or the like of an endoscope cart that holds the control device 11 and the display device 19 and is in a standby state until the examination starts. As described above, although power is supplied to the endoscope 10 and display control is performed, a state in which the endoscope 10 is held at a specific location can also be said to be the non-use state of the endoscope 10. The state in which the endoscope 10 is held at the specific location can be determined based on, for example, a change in the image captured by the endoscope 10, information on the acceleration sensor provided in the endoscope 10, and the like.

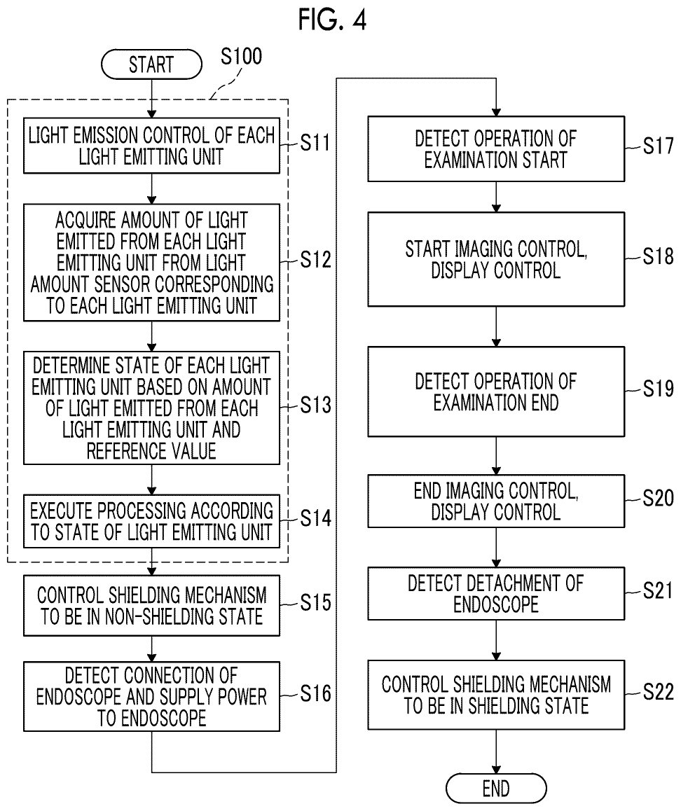

FIG. 4 is a flowchart for describing a processing example (1) of the controller 40. In a case where the power of the control device 11 is turned on and then the controller 40 detects that the endoscope 10 is not connected to the light source unit 30, the controller 40 performs each process shown in FIG. 4. In an initial state immediately after the power of the control device 11 is turned on, it is assumed that the shielding mechanism 38 is in a shielding state.

First, the controller 40 executes state determination processing (step S100) including steps S11 to S14. In step S11, the controller 40 performs the light emission control of causing the light emitting unit 31 to emit light under the first condition. Next, the controller 40 acquires the amount of light emitted by the light emitting unit 31 as the measured light amount P1 from the sensor 32 corresponding to the light emitting unit 31 that emits light in step S11 (step S12). Next, the controller 40 determines the state of the light emitting unit 31 based on the acquired measured light amount P1 and the reference value P2 based on the first condition (step S13). Details of step S13 will be described later. The controller 40 executes the processing of each of the four light emitting units 31 from step S11 to step S13, and then executes the processing according to the determination result for each light emitting unit 31 (step S14). Details of step S14 will be described later.

After step S100, the controller 40 controls the shielding mechanism 38 to be in the non-shielding state (step S15). Next, in a case where the controller 40 detects that the endoscope 10 is connected to the light source unit 30 (Step S16), the controller 40 starts to supply power to the endoscope 10.

Next, in a case where the operation of the examination start button by the user is detected (Step S17), the controller 40 starts the imaging control by the imaging element included in the endoscope 10 and the display control of outputting the image obtained by the imaging control to the display device 19 (Step S18).

Next, in a case where the operation of the examination end button by the user is detected (Step S19), the controller 40 ends the imaging control and the display control (Step S20). Next, the controller 40 detects that the endoscope 10 is detached from the light source unit 30 (Step S21), and controls the shielding mechanism 38 to be in the shielding state (Step S22).

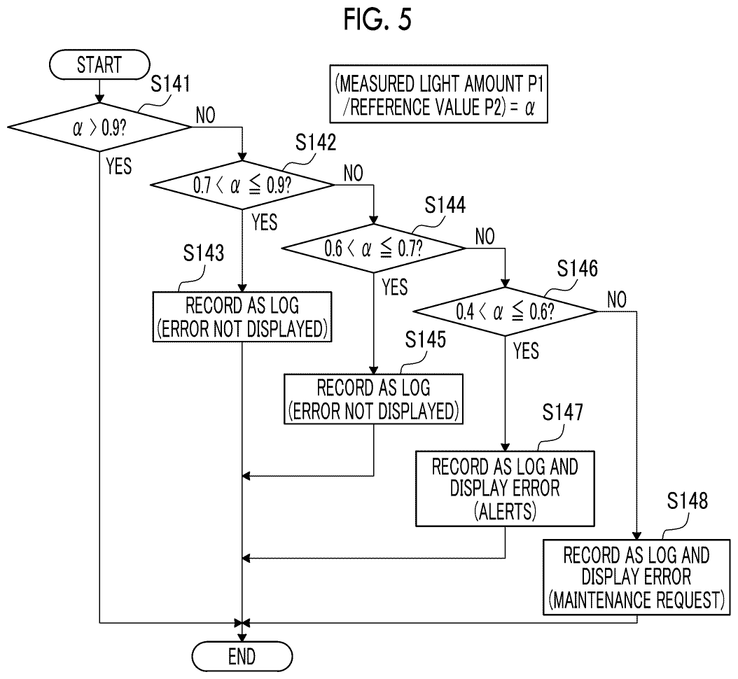

FIG. 5 is a flowchart for describing a specific example of step S13 and step S14 shown in FIG. 4. The processing shown in FIG. 5 is performed for each of the four light emitting units 31.

In step S13, the controller 40 derives a value obtained by dividing the measured light amount P1 by the reference value P2 as an index α indicating the degree of decrease in the light emission amount of the light emitting unit 31 to be determined. Then, the controller 40 determines whether or not the index α exceeds 0.9 (Step S141). In a case where the determination in step S141 is YES, the controller 40 determines that the light emission performance of the light emitting unit 31 to be determined is in the allowable range (the highest in five stages), and ends the state determination processing.

In a case where the index α is 0.9 or less (NO in step S141), the controller 40 determines whether or not the index α exceeds 0.7 (step S142). In a case where the determination in step S142 is YES, the controller 40 determines that the light emission performance of the light emitting unit 31 to be determined is in the allowable range (second from the top among five stages), records the index α, information indicating the stage of the light emission performance, or the like as a log in the memory (step S143), and ends the state determination processing. In step S143, the controller 40 does not output the determination result to the display device 19. In step S143, the controller 40 may transmit the log to a server that manages the endoscope apparatus 2.

In a case where the index α is 0.7 or less (NO in step S142), the controller 40 determines whether or not the index α exceeds 0.6 (step S144). In a case where the determination in step S144 is YES, the controller 40 determines that the light emission performance of the light emitting unit 31 to be determined is in the allowable range (third from the top among five stages), records the index α, information indicating the stage of the light emission performance, or the like as a log in the memory (step S145), and ends the state determination processing. In step S145, the controller 40 does not output the determination result to the display device 19. In step S145, the controller 40 may transmit the log to a server that manages the endoscope apparatus 2.

In a case where the index α is 0.6 or less (NO in step S144), the controller 40 determines whether or not the index α exceeds 0.4 (step S146). In a case where the determination in step S146 is YES, the controller 40 determines that the light emission performance of the light emitting unit 31 to be determined is in the allowable range (fourth from the top among five stages), records the index α, information indicating the stage of the light emission performance, or the like in the memory as a log, and further notifies that the light emission performance of the light emitting unit 31 is deteriorated. For example, the controller 40 outputs and displays first error information based on the index α, information indicating the stage of the light emission performance, or the like on the display device 19 (step S147). Then, the controller 40 ends the state determination processing. The first error information is, for example, a message that alerts that “please note that the light emission amount of the specific light emitting unit 31 is decreasing”.

In a case where the index α is 0.4 or less (NO in step S146), the controller 40 determines that the performance of the light emitting unit 31 to be determined is out of the allowable range (the lowest in five stages), records the index α, information indicating the stage of the light emission performance, or the like in the memory as a log, and further notifies that the use of the light source unit 30 is stopped. For example, the controller 40 outputs and displays second error information based on the index α, information indicating the stage of the light emission performance, or the like on the display device 19 (step S148). Then, the controller 40 ends the state determination processing. The second error information is, for example, a message warning that use is prohibited, such as “The specific light emitting unit 31 is deteriorated. Please stop using it and request maintenance”. It is preferable that the second error information is information indicating that the use of the light source unit 30 is not recommended, compared to the first error information.

In FIG. 5, the processing of step S143 and the processing of step S145 each constitute the first processing. The processing of step S147 and the processing of step S148 each constitute the second processing. A case where a determination result in step S142 is YES and a case where a determination result in step S144 is YES each correspond to a case where the state of the light emitting unit 31 is determined to be the first stage. A case where a determination result in step S146 is YES and a case where a determination result in step S146 is NO each correspond to a case where the state of the light emitting unit 31 is determined to be the second stage (a stage that is not better than the first stage, and a stage in which the degree of decrease of the light emission amount of the light emitting unit 31 is larger than the first stage).

According to the processing example shown in FIG. 4, the state of each light emitting unit 31 in the light source unit 30 is determined in a state where the endoscope 10 is not connected to the light source unit 30. Therefore, it is possible to determine the deterioration or the abnormality of the light emitting unit 31 and to notify the user of the availability of the control device 11 before using the endoscope 10. By performing such determination before the start of the endoscopy, the examination can be efficiently performed. In addition, since the above determination is not performed during the examination of the endoscope (during use), the load on the endoscope apparatus 2 during the examination can be reduced. Further, the first condition can be optionally determined, and the determination accuracy of the state of the light emitting unit 31 can be improved.

In addition, according to the processing example shown in FIG. 4, in a state in which the endoscope 10 is not connected to the light source unit 30, the shielding mechanism 38 is controlled to be in the shielding state. Therefore, even in a case where the processing of step S11 in the state determination processing is performed, the light emitted from the light emitting unit 31 to be determined can be suppressed from being emitted from the connector 12 to the outside. Accordingly, it is possible to determine the state of the light emitting unit 31 without causing the user to feel uncomfortable. In addition, the emission of strong light from the connector 12 can be prevented, and thus safety can be improved. The shielding mechanism 38 is not essential and may be omitted. In this case, in FIG. 4, the processing of step S15 and step S22 may be deleted.

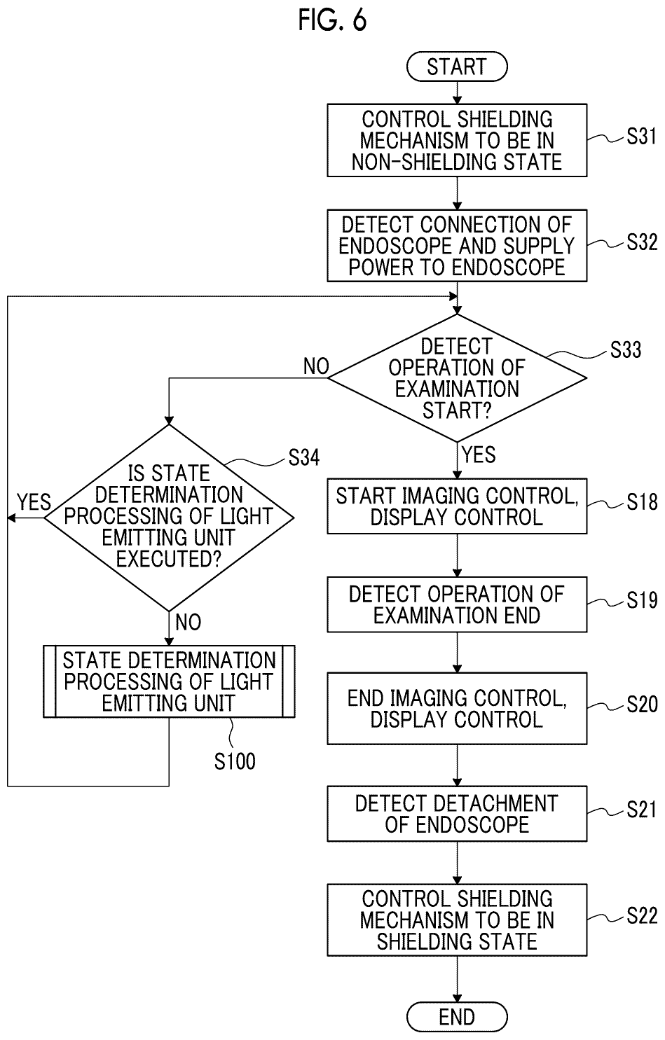

FIG. 6 is a flowchart for describing a processing example (2) of the controller 40. In FIG. 6, the same processing as in FIG. 4 is designated by the same reference numeral, and the description thereof is omitted.

In a case where the power of the control device 11 is turned on, the controller 40 controls the shielding mechanism 38 to be in the non-shielding state (Step S31). Next, in a case where the controller 40 detects that the endoscope 10 is connected to the light source unit 30 (Step S32), the controller 40 starts to supply power to the endoscope 10.

Next, in a case where the operation of the examination start button by the user is detected (Step S33: YES), the controller 40 performs the processing of Step S18 to Step S22. In a case where the operation of the examination start button by the user is not detected (NO in step S33), the controller 40 determines whether or not the state determination processing is executed after the power is turned on (step S34). In a case where the determination in step S34 is NO, the controller 40 executes the state determination processing in step S100. In a case where the determination in step S34 is YES, the controller 40 returns the processing to step S33.

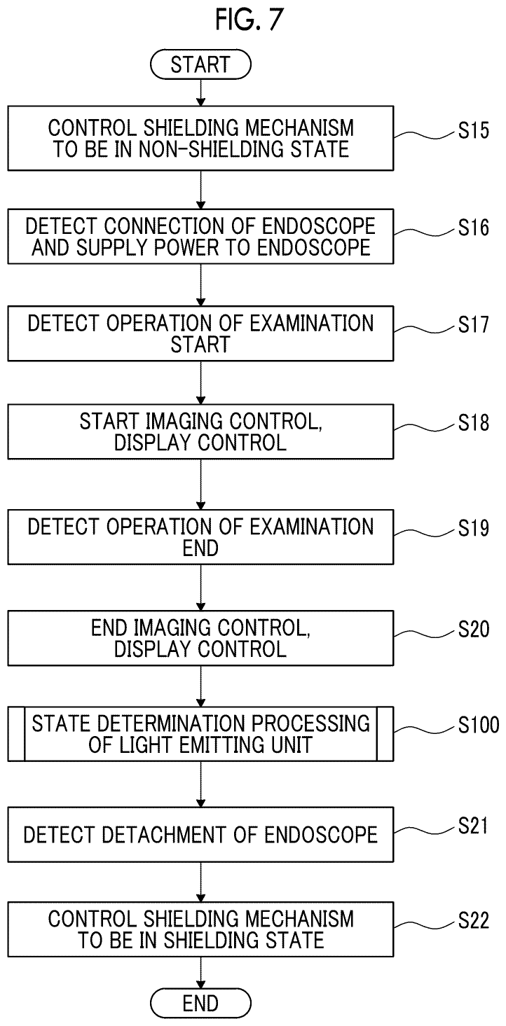

FIG. 7 is a flowchart for describing a processing example (3) of the controller 40. In FIG. 7, the same processing as in FIG. 4 is designated by the same reference numerals, and the description thereof will not be repeated. In a case where the power of the control device 11 is turned on, the controller 40 executes processing of step S15 to step S20, and then performs processing of step S100. After step S100, the controller 40 performs processing of step S21 and step S22.

According to the processing examples shown in FIGS. 6 and 7, the state determination processing is performed before the start of the display control or after the end of the display control in a state where the endoscope 10 is connected to the light source unit 30. Therefore, in a case where the processing of step S11 in the state determination processing is performed, the image displayed on the display device 19 does not flicker. As a result, the state determination processing can be executed without being conscious of the user.

The controller 40 may perform light amount ratio control of causing two or more light emitting units 31 of the plurality of light emitting units 31 to emit light and controlling a ratio of the amounts of light emitted from the two or more light emitting units 31 to a predetermined value. The light amount ratio control is performed by controlling each light emitting unit 31 such that a ratio of the amounts of emitted light measured by the sensor 32 corresponding to each of the two or more light emitting units 31 is a predetermined value.

For example, a case where the B light, G light, V light, and A light are respectively emitted and the light amount ratios of the four colors of light are controlled to predetermined values is assumed. In this case, the target value TG of the measured light amount by the sensor 32G, the target value TB of the measured light amount by the sensor 32B, the target value TV of the measured light amount by the sensor 32V, and the target value TA of the measured light amount by the sensor 32A are set, and the ratio of the target value TG, the target value TB, the target value TV, and the target value TA is the predetermined value.

A case is assumed in which, as a result of the state determination processing, for example, the state of the light emitting unit 31G is third from the top among five stages, and the states of the other light emitting units 31 are the highest among five stages. In this case, the controller 40 decreases the target value TG from the initial value, and decreases the target value TB, the target value TV, and the target value TA from the initial value such that the ratio of the target value TG, the target value TB, the target value TV, and the target value TA is maintained at the predetermined value by the decreased amount. Accordingly, even in a case where the light emitting unit 31G is deteriorated, the ratio of the amounts of light emitted from the four light emitting units 31 can be controlled to the predetermined value. In a case where step S146 is YES or in a case where step S146 is NO, the controller 40 may not execute the above-described light amount ratio control.

In the above description, the amount of emitted light is exemplified as the characteristic value of the light emitting unit 31, but the characteristic value is not limited thereto. For example, the controller 40 may acquire the drive current or the drive voltage measured by the measurement circuit 311A included in the light emitting unit 31 as the characteristic value of the light emitting unit 31, and may determine the state of the light emitting unit 31 based on the characteristic value and the reference value P2.

For example, a case where the controller 40 does not perform the APC control is assumed. In this case, the first condition includes setting a set value of the drive current or the drive voltage of the light emitting unit 31 to any value and driving the light emitting element 310 in accordance with the set value. This set value constitutes the above-described reference value P2. By comparing the reference value P2 with the drive current or the drive voltage measured by the measurement circuit 311A when the light emission control is executed under the first condition, it is possible to determine whether or not there is deterioration or an abnormality in the drive circuit 311 of the light emitting unit 31. For example, in a case where the measured drive current or drive voltage is significantly different from the reference value P2, it can be determined that the drive circuit 311 of the light emitting unit 31 is in a state where deterioration or abnormality has occurred.

Alternatively, the controller 40 may acquire the temperature of the light emitting element 310 measured by the temperature sensor 310A included in the light emitting unit 31 as the characteristic value of the light emitting unit 31, and determine the state of the light emitting unit 31 based on the characteristic value and the reference value P2.

For example, a case where the controller 40 does not perform the APC control is assumed. In this case, the first condition includes causing the light emitting unit 31 to emit light with any amount of emitted light. The value of the temperature measured by the temperature sensor 310A included in the light emitting unit 31 in a case where the light emitting unit 31 is caused to emit light under the first condition is known, and this value constitutes the reference value P2. By comparing the reference value P2 with the temperature measured by the temperature sensor 310A when the light emission control is executed under the first condition, it is possible to determine whether or not the light emitting element 310 of the light emitting unit 31 or the peripheral element thereof is in a state where deterioration or abnormality has occurred. For example, in a case where the temperature measured during the light emission control is significantly higher than the reference value P2, it can be determined that the light emitting element 310 or the peripheral element thereof is in a state where deterioration or abnormality has occurred.

In the present embodiment, each process is executed by any computer. In addition, any computer may execute these processes by a processor, a program, or a combination thereof. Any computer may be a general-purpose computer, a computer for a specific use, a system such as a workstation, or other hardware elements capable of executing a program.

The processor may be configured by one or a plurality of hardware, and the type of hardware is not limited. For example, the processor may be configured by a programmable logic device such as a central processing unit (CPU), a micro processing unit (MPU), or a field programmable gate array (FPGA), a dedicated circuit for executing specific processing such as an application specific integrated circuit (ASIC), or hardware such as a graphic processing unit (GPU) or a neural processing unit (NPU). In addition, the processor has each unit or each means that executes various types of processing in the present embodiment. In addition, the types of hardware may be a combination of different types of hardware. In a case where a plurality of hardware are configured to execute one or a plurality of processes of a certain processor, the plurality of hardware may be present in devices physically separated from each other, or may be present in the same device. In addition, in any of the embodiments, the order of each process by the processor is not limited to the above order and may be appropriately changed. The hardware is configured by an electric circuit (circuitry) in which circuit elements such as semiconductor elements are combined.

Further, the present embodiment may be realized by hardware, software, firmware, microcode, or a combination thereof. Software, firmware, and microcode are configured by a program. In addition, the program may be, for example, a group of program modules, and each function thereof may be realized by a processor configured to execute each function. The program may be a program code or a plurality of code segments stored in one or a plurality of non-transitory computer-readable media (for example, a storage medium or other storage). The program may be divided and stored in a plurality of non-transitory computer-readable media existing in devices physically separated from each other. The program code or the code segment may represent any combination of procedures, functions, subprograms, routines, subroutines, modules, software packages, classes, or commands, data structures, or program statements. The program code or the code segment may be connected to another code segment or a hardware circuit by transmitting and receiving information, data, an argument, a parameter, or a content of a memory.

As described above, at least the following matters are described in the present specification.

(1)

A system comprising:
a light source device including a light emitting unit and a sensor that measures a characteristic value of the light emitting unit, and configured to supply light emitted from the light emitting unit to an endoscope; and
a processor,
wherein the processor is configured to:
   in a first case where the endoscope is not connected to the light source device, or in a second case where the endoscope is connected to the light source device and is in a non-use state,
   perform light emission control of causing the light emitting unit to emit light under a first condition;
   acquire, from the sensor, the characteristic value of the light emitting unit that has emitted light under the light emission control; and
   determine a state of the light emitting unit based on the characteristic value and a reference value.

(2)

The system according to (1),
wherein the processor is configured to perform display control of outputting an image captured by the endoscope to a display device, and
the non-use state includes a state in which the display control is not performed.

(3)

The system according to (1) or (2),
wherein the non-use state includes a state in which power is not being supplied to the endoscope.

(4)

The system according to any one of (1) to (3),
wherein the processor is configured to:
   perform the light emission control in the first case;
   acquire, from the sensor, the characteristic value of the light emitting unit that has emitted light under the light emission control; and
   determine the state of the light emitting unit based on the characteristic value and the reference value.

(5)

The system according to any one of (1) to (4),
wherein the light emitting unit includes a plurality of light emitting units,
the system further includes a shielding mechanism configured to shield at least a part of light combined and emitted from the plurality of light emitting units, and
the first condition includes that the light emitted from the light emitting unit is shielded by the shielding mechanism.

(6)

The system according to (5),
wherein the processor is configured to,
   in a case where the endoscope transitions from a state of being connected to the light source device to a state of not being connected to the light source device,
   control the shielding mechanism into a state in which the light is shieldable.

(7)

The system according to any one of (1) to (6),
wherein the state of the light emitting unit is a degree of decrease in light emission amount.

(8)

The system according to any one of (1) to (7),
wherein the characteristic value is the light emission amount.

(9)

The system according to (8),
wherein the processor is configured to:
   acquire the light emission amount from the sensor; and
   perform light amount control of bringing the light emission amount closer to a set light amount.

(10)

The system according to (9),
wherein the first condition includes setting a set value of the light emission amount of the light emitting unit to a value equal to or greater than 80% of an upper limit value settable by the light emitting unit.

(11)

The system according to (10),
wherein the first condition includes setting the set value to the upper limit value.

(12)

The system according to any one of (7) to (11),
wherein the light emitting unit includes a plurality of light emitting units, and
the processor is configured to perform, based on the degree of decrease in the light emission amount of the light emitting unit, light amount ratio control of causing two or more of the plurality of light emitting units to emit light and controlling a ratio of the light emission amount of the two or more light emitting units to a predetermined value.

(13)

The system according to any one of (1) to (12),
wherein the processor is configured to:
   determine the state of the light emitting unit in a plurality of stages; and
   perform processing that differs based on the determined stage.

(14)

The system according to (13),
wherein the processing includes at least one of first processing of recording information based on the determined state, or second processing of recording and notifying the information.

(15)

The system according to (14),
wherein the processor is configured to:
   perform the first processing in a case where the state of the light emitting unit is determined to be a first stage; and
   perform the second processing in a case where the state of the light emitting unit is determined to be a second stage in which the degree of decrease in the light emission amount of the light emitting unit is greater than that in the first stage.

(16)

The system according to (15),
wherein the second stage is further divided into a plurality of stages, and
a content of the notification in the second processing is different for each of the plurality of stages.

(17)

The system according to (16),
wherein the light amount ratio control of causing two or more of the plurality of light emitting units to emit light and controlling a ratio of the light emission amount of the light emitting units to a predetermined value is not executed, in a case where the state of the light emitting unit is determined to be a stage in which the degree of decrease in the light emission amount of the light emitting unit exceeds a predetermined value among the plurality of stages.

### EXPLANATION OF REFERENCES

2: endoscope apparatus
10: endoscope
11: control device
12: connector
13: insertion part
14: distal end part
15: operation part
17: universal cord
18: endoscope connector
19: display device
20: light guide rod
30: light source unit
31, 31A, 31B, 31G, 31V: light emitting unit
32, 32A, 32B, 32G, 32V: sensor
33A, 33B, 33G, 33V: half mirror
34, 35, 36: dichroic mirror
37: housing
37A: opening
38: shielding mechanism
38A: shutter member
40: controller
310: light emitting element
310A: temperature sensor
311: drive circuit
311A: measurement circuit
312: phosphor

What is claimed is:

1. A system comprising:
a light source device including a light emitting unit and a sensor that measures a characteristic value of the light emitting unit, and configured to supply light emitted from the light emitting unit to an endoscope; and
a processor,
wherein the processor is configured to:
in a first case where the endoscope is not connected to the light source device, or in a second case where the endoscope is connected to the light source device and is in a non-use state,
perform light emission control of causing the light emitting unit to emit light under a first condition;
acquire, from the sensor, the characteristic value of the light emitting unit that has emitted light under the light emission control; and
determine a state of the light emitting unit based on the characteristic value and a reference value.

2. The system according to claim 1,
wherein the processor is configured to perform display control of outputting an image captured by the endoscope to a display device, and
the non-use state includes a state in which the display control has not been performed.

3. The system according to claim 2,
wherein the non-use state includes a state in which power has not been supplied to the endoscope.

4. The system according to claim 1,
wherein the processor is configured to:
perform the light emission control in the first case;
acquire, from the sensor, the characteristic value of the light emitting unit that has emitted light under the light emission control; and
determine the state of the light emitting unit based on the characteristic value and the reference value.

5. The system according to claim 4,
wherein the light emitting unit includes a plurality of light emitting units,
the system further includes a shielding mechanism configured to shield at least a part of light combined and emitted from the plurality of light emitting units, and
the first condition includes shielding the light emitted from the light emitting unit by the shielding mechanism.

6. The system according to claim 5,
wherein the processor is configured to,
in a case where the endoscope transitions from a state of being connected to the light source device to a state of not being connected to the light source device,
control the shielding mechanism into a state in which the light is shieldable.

7. The system according to claim 1,
wherein the state of the light emitting unit is a degree of decrease in light emission amount.

8. The system according to claim 7,
wherein the characteristic value is the light emission amount.

9. The system according to claim 8,
wherein the processor is configured to:
acquire the light emission amount from the sensor; and
perform light amount control of bringing the light emission amount closer to a set light amount.

10. The system according to claim 9,
wherein the first condition includes setting a set value of the light emission amount of the light emitting unit to a value equal to or greater than 80% of an upper limit value settable by the light emitting unit.

11. The system according to claim 10,
wherein the first condition includes setting the set value to the upper limit value.

12. The system according to claim 7,
wherein the light emitting unit includes a plurality of light emitting units, and
the processor is configured to perform, based on the degree of decrease in the light emission amount of the light emitting unit, light amount ratio control of causing two or more of the plurality of light emitting units to emit light and controlling a ratio of the light emission amount of the two or more light emitting units to a predetermined value.

13. The system according to claim 12,
wherein the processor is configured to:
determine the state of the light emitting unit from a plurality of stages; and
perform processing that differs based on the determined stage.

14. The system according to claim 13,
wherein the processing includes at least one of first processing of recording information based on the determined state, or second processing of recording and notifying the information.

15. The system according to claim 14,
wherein the processor is configured to:
perform the first processing in a case where the state of the light emitting unit is determined to be a first stage; and
perform the second processing in a case where the state of the light emitting unit is determined to be a second stage in which the degree of decrease in the light emission amount of the light emitting unit is greater than that in the first stage.

16. The system according to claim 15,
wherein the second stage is further divided into a plurality of stages, and
a content of the notification in the second processing is different for each of the plurality of stages.

17. The system according to claim 16,
wherein the light amount ratio control is not executed in a case where the state of the light emitting unit is determined to be a stage in which the degree of decrease in the light emission amount of the light emitting unit exceeds a predetermined value among the plurality of stages.
